(19) United States
(12) Patent Application Publication
(10) Pub. No.: US 2005/0228066 A1
(43) Pub. Date: Oct. 13, 2005

(54) Film extruded denture adhesive liner

(75) Inventors: Eddie Wong, New Providence, NJ (US); Robert C. Gasman, Montville, NJ (US); Johny Ortiz, Elizabeth, NJ (US)

Correspondence Address: GLAXOSMITHKLINE;Corporate Intellectual Property, UW2220, P.O. Box 1539, King Of Prussia, PA, 19406-0939, US

(73) Assignee: Block Drug Co. Inc.

(21) Appl. No.: 11/150,753

(22) Filed: Jun. 10, 2005

Related U.S. Application Data
(63) Continuation of application No. 10/398,530, filed on Apr. 8, 2003, now abandoned.
Application No. 10/398,530 is a continuation of application No. 09/686,787, filed on Oct. 10, 2000, now abandoned.

(30) Foreign Application Priority Data
Oct. 10, 2000 (US) 09686787

(51) Int. Cl. A61K 6/00

(52) U.S. Cl. 523/120

(57) ABSTRACT

A denture adhesive liner in the form of an extruded film or sheet comprising (a) a denture adhesive effective amount of a mixed partial salt of a copolymer of maleic acid and an alkyl vinyl ether and at least one cation, wherein all of said cations are selected from the group consisting of sodium, potassium, calcium, strontium, magnesium, zinc and zirconium oxy cations; (b) between about 30 and 90 wt. % of a thermoplastic polymer component; and (c) a plasticizer; wherein said composition is extrudable into a film that is capable of adhering to a wet mucous surface and/or acrylic plastic.

### FIELD OF THE INVENTION

The present invention relates to an adhesive liner for dental prosthesis in the form of an extruded film.

### BACKGROUND OF THE INVENTION

Dentures are substitutes for missing teeth and serve as replacement for all or some of the teeth found in the oral cavity. Over time, even well fitting dentures can become ill fitting due to natural shrinkage and changes in the gum or mucous tissues. Therefore, adherent creams, liquids, powders, and “liners” are often used to secure dentures within the mouth. Liners are denture adhesives in the form of a thin film, strip, or wafer with a certain desirable strength and integrity for the liner to be placed in between the prosthesis and the palate or jaw, which swells in the mouth fluid without using a support.

Denture adhesive liners disclosed in the prior art are commonly in the form of a woven composite or a multiple layer strip. U.S. Pat. No. 3,990,149 discloses an adhesive foil comprising a compress fiber mat. U.S. Pat. No. 4,880,702 describes a denture stabilizer in the form of a strip consisting of three different layers. U.S. Pat. No. 5,158,825 discloses a denture liner in the form of a non-woven fabric which is impregnated with an adhesive. U.S. Pat. No. 4,503,116 discloses a denture adhesive liner comprising a laminate of superimposed fiber faced webs, with the fibers of one face of the webs being heat bonded to the fibers on the opposing webs by thermoplastic ethylene oxide polymers. U.S. Pat. No. 5,877,233 discloses a multi-layer denture adhesive liner with at least one non-adhesive self-supporting layer coated by adhesive components.

U.S. Pat. No. 4,373,036 discloses a denture fixative in the form of a single-layer strip or film. The single-layer denture adhesive liner disclosed is prepared using a film casting method under vacuum at 55±5° C. from a composition of 43.6 parts of a partially neutralized copolymer of ethyl vinyl ether-maleic anhydride, 64.4 parts of hydroxypropyl cellulose, 1 part of color, flavor, antioxidant and preservatives for a total of 120 parts, which is then mixed with 472 parts of water, and 8 parts of glycerin forming a viscous solution. This approach is not economically attractive owing to the amount of time and energy needed to evaporate the water inherently required in the composition to dissolve the hydroxypropyl cellulose into a solution that can be cast onto a moving carrier tape by means of a die, thereafter forming a film that can be dried under vacuum at high temperatures.

### SUMMARY OF THE INVENTION

The present invention is directed to a denture adhesive liner in the form of an extruded film or sheet.

The present invention provides a denture adhesive liner in the form of an extruded film or sheet, comprising: (a) a denture adhesive effective amount of a mixed partial salt of a copolymer of maleic acid and an alkyl vinyl ether and at least one cation, wherein all of said cations are selected from the group consisting of sodium, potassium, calcium, strontium, magnesium, zinc and zirconium oxy cations; (b) 30-90 wt. % of a thermoplastic polymer component; and (c) a plasticizer, wherein said composition is extrudable into a film that is capable of adhering to a wet mucous surface.

In one embodiment of the present invention, the thermoplastic polymer component is selected from the group consisting of a polyethylene oxide polymer having a weight average molecular weight that is between about 100,000 to about 20,000,000, hydroxy propyl cellulose, and hydroxy propyl methylcellulose or mixtures thereof.

The present invention also provides a method for preparing a denture adhesive liner comprising the steps of:
(a) preparing a composition of a denture adhesive effective amount of a mixed partial salt of a copolymer of maleic acid and an alkyl vinyl ether and at least one cation, wherein all of said cations are selected from the group consisting of sodium, potassium, calcium, magnesium, zinc and zirconium cations; a plasticizer; and 40-90 wt. % of a thermoplastic polymer component, said thermoplastic component selected from the group consisting of a polyethylene oxide polymer having a weight average molecular weight that is between about 100,000 to about 20,000,000, hydroxy propyl cellulose, and hydroxy propyl methyl cellulose or mixtures thereof; and
(b) forming a denture adhesive liner from said composition by extruding said composition under increased pressure through a die such that it forms a film.

### DESCRIPTION OF THE DRAWING

FIG. 1 is a graph plotting adhesive force in lbs. vs. the hydration time in minutes, comparing the adhesive composition of the invention with comparative examples.

### DETAILED DESCRIPTION OF THE INVENTION

The first component of the present invention is a partial salt of a copolymer of maleic acid or maleic anhydride and a lower alkyl vinyl ether (“PVM/MA”). In one embodiment, the alkyl group is from 1 to about 5 carbon atoms. Lower alkyl vinyl ether maleic polymers are readily obtained by copolymerizing a lower alkyl vinyl ether monomer, such as methyl vinyl ether, ethyl vinyl ether, divinyl ether, propyl vinyl ether, isobutyl vinyl ether, and the like, with maleic anhydride to yield the corresponding lower alkyl vinyl ether-maleic anhydride copolymer which is readily hydrolyzable to the acid copolymer. In general, the resulting copolymer is a 1:1 copolymer. Both anhydride and acid forms are also available from commercial suppliers, e.g. ISP Technologies Inc. (“ISP”), Wayne, N.J., U.S.A.

The partial copolymer salts of the present invention comprise cationic salt function, with the cations being one or more metal cations selected from the group consisting of divalent cations, monovalent cations, and mixtures thereof. Divalent metal cations include zinc, strontium (but not used in combination with zinc), calcium, magnesium and mixtures thereof. Monovalent metal cations include sodium, potassium, ammonium, and mixtures thereof. The copolymer salts may be mixed or unmixed or both. The term “unmixed polymer salts” as used herein refers to salts of lower alkyl vinyl ether-maleic polymers wherein the cations are unmixed with any other ester functions or non-identical cations on the same polymer, the remaining carboxyl groups being unreacted. The term “mixed polymer salts” as used herein refers to salts of the lower alkyl vinyl ether-maleic polymers where different cations are mixed on the same polymer with each other or with other ester functions.

Partial copolymer salts comprising divalent and/or monovalent metal cations can be prepared by the interaction of the PVM/MA anhydride/acid copolymers with metal cation compounds (such as zinc, strontium, calcium, magnesium, sodium, potassium, or ammonium) either in the form of a base or a salt; such as, for example, the hydroxide, acetate, halide, lactate, etc., in an aqueous medium. Examples are single or mixed salts of calcium/sodium, calcium/potassium, zinc/magnesium, and sodium/zinc/magnesium. Exemplary mixed salts of two cations (“double salts”) included within the scope of this invention are calcium/sodium, calcium/magnesium, calcium/zinc, sodium/zinc, potassium/zinc, sodium/magnesium, potassium/mangnesium, calcium/potassium or zinc/magnesium salts. Exemplary mixed salts of three cations (“triple salts”) included within the scope of this invention are calcium/sodium/zinc or sodium/zinc/magnesium salts.

When the salts are prepared, the metal compounds react with the carboxylic acid groups on the copolymer and neutralize them. Preferably less than 100% of the carboxylic acid groups on the copolymer chain are neutralized. In one embodiment, between about 50 to 90% of the carboxylic acid groups of the copolymer are neutralized. In another embodiment, about 65 to 75% of the carboxylic acid groups are neutralized.

The first component of the present invention is present in a denture adhesive effective amount. A denture adhesive effective amount means an amount sufficient for a flexible and uniform denture adhesive liner product with good denture adhesive properties, e.g., exhibiting a sufficient cohesive strength to withstand the stresses of mastication which act to rupture the seal and thus dislodge the denture; resistance to degradation under the extreme environmental changes that occur in the oral cavity during such common actions as drinking coffee or other hot beverages; and releasable properties so that the denture wearer may remove the dentures for cleaning and maintenance.

In one embodiment, the partial salt of a copolymer of maleic acid and a lower alkyl vinyl ether is present in an amount from about 5 to 55 wt. %. In another embodiment, the amount is about 10 to 25 wt. %.

The second component is a thermoplastic polymer which is water soluble. A “thermoplastic polymer” is meant to refer to a material which is melt processable. As used herein, the term thermoplastic refers to a material which softens and/or becomes flexible when exposed to heat and generally returns to its original condition when cooled to room temperature.

As used herein, a material will be considered to be water soluble when it substantially dissolves in excess water to form a solution, thereby losing its initial form and becoming essentially molecularly dispersed throughout the water solution. As a general rule, a water-soluble material will be free from a substantial degree of cross-linking, as cross-linking tends to render a material water insoluble. Also used herein, the term “water-insoluble” is meant to refer to a material that, when exposed to an excess of water, disperses but does not dissolve. As such, a water-insoluble material generally retains its original identity or physical structure, but in a highly dispersed state and must have sufficient physical integrity to resist flow and fusion with neighboring materials.

In one embodiment, the thermoplastic polymer component is selected from polyethylene oxide polymer, hydroxy propyl cellulose, hydroxy propyl methyl cellulose, or mixtures thereof.

If polyethylene oxide polymer (“PEO”) is used, it is desired that the material exhibits a weight average molecular weight that is effective for the denture adhesive liner composition to exhibit sufficient cohesive strength and resistance to degradation properties. In general, if the weight average molecular weight of a PEO polymer is too high, the polymer chains may become heavily entangled which may result in a thermoplastic composition which is difficult to process. In one embodiment, the PEO polymers suitable for use in the present invention exhibit weight average molecular weights between about 100,000 to about 20,000,000. In another embodiment, the molecular which are between about 200,000 to about 8,000,000.

In one embodiment, PEO is present in the denture adhesive liner composition of the present invention in an amount between 0 and 90 wt. %. In another embodiment, PEO is present in an amount between 50 and 90 wt. %. In a third embodiment, PEO is present between 30 and 70 wt. % in combination with another thermoplastic polymer. In yet a fourth embodiment, PEO is present as the only thermoplastic polymer in an amount between 30 and 90 wt. %.

Hydroxy propyl cellulose (“HPC”) polymers having a weight average molecular weight between 80,000-1,150,000 are useful for the purposes of this invention. HPC can be used as a thermoplastic polymer component by itself or in combination wit other thermoplastic polymer components, i.e., PEO, and the like. HPC is commercially available from Hercules, Inc. (Wilmington, Del.) under the trade name KLUCEL.

In one embodiment, HPC is present in the denture adhesive liner composition of the present invention in an amount between 0 and 90 wt. %. In another embodiment, HPC is present between 5 and 20 wt. % in combination with another thermoplastic polymer. In yet a third embodiment, HPC is present as the only thermoplastic polymer in an amount between 60 and 90 wt. %.

Hydroxy propyl methyl cellulose (“HPMC”) is another water-soluble cellulose that exhibits thermoplastic polymer processing properties when used in combination with a plasticizer. HPMC can be used as a thermoplastic polymer component by itself or in combination with other thermoplastic polymer components, i.e., PEO, and the like. HPMC is commercially available from Dow Chemical Company of Midland, Mich., USA, under the trade name METHOCEL, which is a HPMC which is a 2% concentration in water produces a viscosity of 400 cPs.

In one embodiment, HPMC is present in the denture adhesive liner composition of the present invention in an amount between 0 and 90 wt. %. In another embodiment, HPMC is present between 5 and 20 wt. % in combination with another thermoplastic polymer. In yet a third embodiment, HPMC is present as the only thermoplastic polymer in an amount between 60 and 90 wt. %.

It is generally desired that a toxicologically acceptable compatibilizer or plasticizer be used as an optional third component in an amount sufficient for the denture adhesive composition to exhibit desired extrusion processability properties. The term “toxicologically acceptable”, as used herein, describes materials which are suitable in their toxicity profile for administration to humans and/or lower animals.

Suitable plasticizers include water, polyethyleneoxide; polypropyleneoxide; glycols such as propylene glycol and polyethylene glycol; polyhydrix alcohols such as glycerin and sorbitol; glycerol esters such as glycerol triacetate; fatty acid triglycerides; naphthenic oils; aromatic oils; vegetable oils such as castor oil; or low molecular weight rosin esters, polyterpenes, and the like.

If PEO is used as a thermoplastic polymer by itself, it has been suggested that water may be used as a fugitive plasticizer for PEO during melt processing.

If HPMC is used as a thermoplastic polymer by itself, it has been suggested that propylene glycol may be used as a plasticizer during melt processing.

Plasticizer may be present at a level of from about 0 to about 30 wt. %. In one embodiment, plasticizer is included in an amount of about 5 to about 25 wt. %.

Optionally, the denture adhesive liner composition may comprise one or more therapeutic actives suitable for mucosal or topical administration. The phrase “suitable for mucosal or topical administration,” as used herein, describes agents which are pharmacologically active when absorbed through internal mucosal surfaces of the body such as the oral cavity, or applied to the surfaces of the skin. Therapeutic actives may be present at a level of from about 0 to about 30 wt. % of the total composition.

Therapeutic activities that are useful in these compositions include antimicrobial agents such as iodine, sulfonamides, bisbiguanides, or phenolics; antibiotics such as tetracycline, neomycin, kanamycin, metronidazole, or clindamycin; anti-inflammatory agents such as aspirin, acetaminophen, naproxen and its salts, ibuprofen, ketorolac, flurbiprofen, indomethacin, cimetidine, eugenol, or hydrocortisone; dentinal desensitizing agents such as potassium nitrate, potassium chloride, strontium chloride or sodium fluoride; anesthetic agents such as lidocaine or benzocaine; anti-fungals; aromatics such as camphor, eucalyptus oil, and aldehyde derivatives such as benzaldehyde; insulin; steroids; and anti-neoplastics. It is recognized that in certain forms of therapy, combinations of these agents in the same delivery system may be useful in order to obtain an optimal effect. Thus, for example, an antimicrobial and an anti-inflammatory agent may be combined in a single delivery system to provide combined effectiveness.

The composition may also comprise other suitable ingredients including silicon dioxide, colorants, preservatives such as methyl and propyl parabens, thickeners, and the like. Preferred are polyethylene glycol, silicon dioxide, and petrolatum. Colorants, preservatives, thickeners and delivery vehicles may be present at levels of from about 0 to about 20 wt. % of the total composition.

The compositions of the present invention may also include one or more components which provide flavor, fragrance, and/or sensate benefit. Suitable components include natural or artificial sweetening agents, menthol, menthyl lactate, wintergreen oil, peppermint oil, spearmint oil, leaf alcohol, as well as coolants 3-1-menthoxypropane-1,2-diol and paramenthane carboxyarnide agents such as N-ethyl-p-menthane-3-carboxamide. These agents may be present at a level of from about 0 to about 30 wt. % of the total composition.

The present compositions can be prepared by any of the methods or combination of methods which follow. The term “mixture,” as used herein, refers to a solution, slurry, or suspension.

The lower alkyl vinyl ether maleic anhydride copolymers can be obtained either from commercial suppliers or by copolymerization of a lower alkyl vinyl ether monomer with maleic anhydride to yield the corresponding lower alkyl vinyl ether-maleic anhydride copolymer which is readily hydrolyzable to the acid copolymer. Covalent cross-linking of PVM/MA copolymer may be achieved though esterification of the maleic anhydride unit of the copolymer. The polymer may also be cross-linked with polyvalent metal ions in addition to the polyol during or after esterification. The resultant polymer may be dried either in a forced air mechanical convection oven or a drum dryer. After drying, the sticky polymer turns into brittle flakes which can be peeled off from the drying surface and further grounded to a fine powder as desired.

In one embodiment, partial salt of a copolymer of maleic acid and a lower alkyl vinyl ether is mixed together with the film forming component(s) and the optional components in a high shear mixer. Plasticizer is gradually added until a free flowing mixture if formed. Mixing can also be done directly in the extruder such as a twin-screw extruder. The mixture is fed into an extruder, e.g., a co-rotating twin screw or single-stage extruder pre-heated to between 70 and 120° C. and then extruded through a die preset at a certain mil gap, e.g., ten mil gap. The product extruded film may be pressed smooth in a hydraulic press or flat-roller or other suitable means, then die-cut to desired shape and size with a stamping machine.

Suitably the denture liner of the present invention may be extruded as a multi-layer film using a multi-layer co-extrusion machine. However, while possible, this is not necessary considering the additional costs. The resulting product has one or more layers being the denture liner composition of the present invention, and one or more layers prepared from a non-adhesive material such as plastic, microcrystalline wax, cloth, fleece, and the like.

The following examples further describe and demonstrate embodiments within the scope of the present invention. The examples are given solely for the purpose of illustration and are not to be construed as limitations of the present invention.

### EXAMPLES

In Examples IV, V, and VI wherein partial salts of a PVM/MA were used, the salts were prepared in the following manner. 900 g of room temperature water was charged into a main reactor kettle equipped with a high speed stirrer. The anhydrous PVM/MA copolymer was slowly added to the main mix kettle with continuous mixing. 250 g of room temperature water was charged into a secondary kettle and sodium hydroxide, magnesium oxide, and zinc oxide were added slowly. This slurry was well mixed to form a homogenous slurry. The slurry was slowly added into the main reactor kettle while mixing at high speed to prevent localized precipitation. The batch was heated to 85° C. (±5° C.) and maintained at about 85° C. for two hours with vigorous mixing, forming the salt. These salts remained in solution and did not precipitate or settle. The resulting mixture was put in trays and dried at 85° C. in an oven or dried on a drum drier. The dried Na/Mg/Zn, 10%/ /20%/ /40% degree of substitution salt was then milled through a suitable mill and screened through a 60 mesh screen. A one percent solution of the resulting powder had a pH of about 5.5-6.5 and a bulk density of 0.7-0.8.

In all examples, all ingredients were blended using a high shear (e.g., Henschel type) mixer for about five minutes. Plasticizer was added slowly to the mixer until a free flowing mixture is formed. The product was then fed into a co-rotating twin screw extruder which was preheated to between 70 and 120° C., and then extruded through a die set at ten rail gap. The extruder film was then die-cut to denture shape and size with a stamping machine.

The materials used and the amounts used for the Examples are set forth (as weight percent) in the table which follows.

| Ingredients | Ex. I | Ex. II | Ex. III | Ex. IV | Ex. V | Ex. VI |
|---|---|---|---|---|---|---|
| Polyethylene oxide | 77.176 | 66.152 | 66.152 | 48.0 | 48.0 | 0 |
| Hydroxy Propyl Cellulose | 0 | 0 | 11.024 | 5.336 | 5.336 | 82.5 |
| Sodium carboxy methyl | 0 | 11.024 | 0 | 2.664 | 2.664 | 0 |
| cellulose | | | | | | |
| Fumed Silica | 2.824 | 2.824 | 2.824 | 2.664 | 2.664 | 2.5 |
| Plasticizer (water) | 20.0 | 20.0 | 20.0 | 20.0 | 20.0 | 2.5 |
| Plasticizer-Polyethylene | 0 | 0 | 0 | 0 | 0 | 2.5 |
| glycol | | | | | | |
| Starch | 0 | 0 | 0 | 5.336 | 3.20 | 0 |
| Na/Ca partial salt of | 0 | 0 | 0 | 16.0 | 16.0 | 10.0 |
| PVM/MA | | | | | | |
| Sodium Citrate | 0 | 0 | 0 | 0 | 2.136 | 0 |

In organoleptic evaluation tests, several evaluation experts were presented with denture adhesive liner samples prepared from Examples I-VI. The evaluation experts were asked to evaluate the denture adhesive liners based on different criteria including mouth feel, hold property (cohesive strength to withstand the stresses of mastication which act to rupture the seal and thus dislodge the denture), time for hold properties to develop, and resistance to degradation (under environmental changes that occur in the oral cavity during such common actions as drinking coffee or other hot beverages). Feedback and results of the tests indicate that the denture liners prepared from Examples IV, V, and VI were judged to provide superior denture stabilizer properties compared to the denture liners of Examples I-III, which were prepared without the partial salts of a lower PVM/MA.

Adhesive Force Evaluation

In this evaluation, denture adhesive liners prepared using the formulation in Example IV were compared with two commercial denture adhesive liners Seabond® and TouchCorrect®. Seabond® is available from Combe Inc. of White Plains, N.Y., USA, the assignee of U.S. Pat. Nos. 4,503,116; 4,632,880; and 5,624,745 disclosing denture adhesive liners comprising PEO powder and other additives between webs of cellulose acetate fibers. TouchCorrect® is available from Shionogi & Co., Ltd., of Osaka, Japan, the assignee of U.S. Pat. No. 4,880,702, disclosing a denture liner consisting of three layers, two outside layers consisting of PEO, sodium carboxymethyl cellulose and polyvinyl alcohol, and an inside layer consisting essentially of microcrystalline wax and PEO.

The four liners were evaluated for adhesion characteristics by an adhesive force testing method using a Chatillon® gauge, Model TCM 201 or 2731-6 with a DP-50 gauge, commercially available from John Chatillon and Sons, New York, N.Y. The Chatillon® instrument measures the force required to separate the tested material from the acrylic plate (and/or metal plate covered with cloth material) to which the test materials are adhered.

In this adhesion force test, 0.15 g of a liner sample was placed on the lower plate of the Chatillon gauge covered with a piece of cloth dampened with 1 mL of deionized water. For the first test point, a compression force of 20 lbs. was applied for 5 minutes. Then the lower plate was pulled away at a rate of 0.7 in./min. The amount of force applied to separate the plates was taken as the measure of the adhesion strength of the liner.

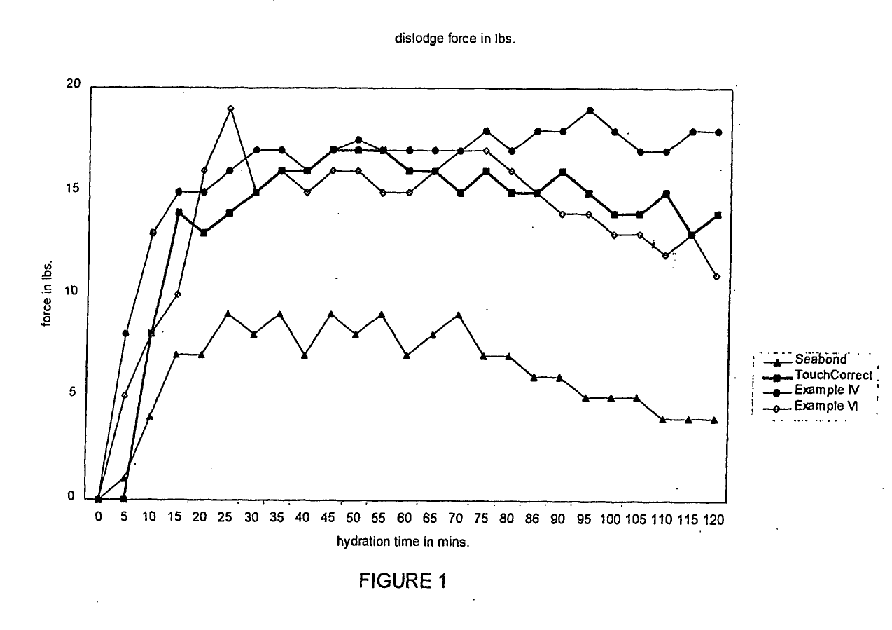

The test was repeated for a time period in 5 minute increments, i.e., 10 minutes, then 15 minutes, . . . , up to 120 minutes. Adhesion strength in lbs. was recorded and then plotted graphically in FIG. 1. The higher the force required to separate the hydrated denture liner sandwiched between the two plates, the better the denture adhesive performance.

The adhesive force evaluation test indicates that the denture adhesive liners prepared from the composition of the present invention using a simple extrusion process are comparable, fi not superior to commercially available denture liners prepared using multiple-layer format/processing techniques.

The above description fully discloses the invention including preferred embodiments thereof. Modifications and improvements of the embodiments specifically disclosed herein are within the scope of the following claims. Without further elaboration it is believed that one skilled in the art can, given the preceding description, utilize the present invention to its fullest extent. Therefore any examples are to be construed as merely illustrative and not a limitation on the scope of the present invention in any way. The embodiments of the invention in which an exclusive property or privilege is claimed are defined as follows.

1. A denture adhesive liner in the form of an extruded film or sheet comprising:
(a) a denture adhesive effective amount of a mixed partial salt of a copolymer of maleic acid and an alkyl vinyl ether and at least one cation, wherein all of said cations are selected from the group consisting of sodium, potassium, calcium, magnesium, zinc and zirconium oxy cations;
(b) a thermoplastic polymer component; and
(c) a plasticizer;
wherein said composition is extrudable into a denture adhesive liner in the form of a firm or sheet that is capable of adhering to a wet mucous surface.

2. The denture adhesive liner of claim 1, wherein said thermoplastic polymer component is selected from the group consisting of a polyethylene oxide polymer having a weight average molecular weight that is between about 100,000 to about 20,000,000, hydroxy propyl cellulose, hydroxy propyl methyl cellulose, and mixtures thereof.

3. The denture adhesive liner of claim 1 in the form of a single-layer extruded film or sheet.

4. The denture adhesive liner of claim 1, wherein said mixed partial salt of a copolymer of maleic acid and an alkyl vinyl ether and at least one cation is present in an amount of about 5 to 55 wt. %, based on the total weight of the denture adhesive liner composition.

5. The denture adhesive liner of claim 1, wherein said plasticizer is present in an amount of from 0 to about 30 wt. %, based on the total weight of the denture adhesive liner composition.

6. The denture adhesive liner of claim 1, wherein said thermoplastic polymer component is present in an amount of from about 30 to 90 wt. %, based on the total weight of the denture adhesive liner composition.

7. The denture adhesive liner of claim 2, further including from about 0 to about 50 wt. %, based on the total weight of the denture adhesive composition, of additional materials selected from the group consisting of sodium carboxymethyl cellulose, polyvinyl pyrrolidone, polyvinyl alcohol, polyacrylic acid derivatives, sodium alginate, polyvinyl acetate, and mixtures thereof.

8. The denture adhesive liner of claim 7, further including additional materials selected from the group consisting of waxes, preservatives, flavoring agents, colorants, sweetening agents, viscosity modifiers, and mixtures thereof.

9. The denture adhesive liner of claim 2, wherein said thermoplastic polymer component is selected from the group consisting of a polyethylene oxide polymer having a weight average molecular weight that is between about 100,000 and about 20,000,000.

10. The denture adhesive liner of claim 1, wherein said thermoplastic polymer component is a mixture of hydroxy propyl cellulose and a polyethylene oxide polymer having a weight average molecular weight that is between about 100,000 and about 20,000,000.

11. A method for preparing a denture adhesive liner comprising the steps of:
(a) preparing a composition comprising: (i) a denture adhesive effective amount of a mixed partial salt of a copolymer of maleic acid and an alkyl vinyl ether and at least one cation, wherein all of said cations are selected from the group consisting of sodium, potassium, calcium, strontium, magnesium, zinc and zirconium oxy cations; (ii) a thermoplastic polymer component; and (iii) a plasticizer; and
(b) forming a denture adhesive liner from said composition by extruding said composition through an extruder under increased pressure through a die such that it forms a film or sheet.

12. The method of claim 11, wherein said denture adhesive liner is in the form of a single-layer extruded film or sheet.

13. The method of claim 11, wherein said denture adhesive liner is extruded using a co-extruder forming a multi-layer extruded film.

14. The method of claim 11, wherein said thermoplastic polymer component is selected from the group consisting of a polyethylene oxide polymer having a weight average molecular weight that is between about 100,000 and about 20,000,000, hydroxy propyl cellulose, hydroxy propyl methyl cellulose, and mixtures thereof.

15. The method of claim 11, wherein said a mixed partial salt of a copolymer of maleic acid and an alkyl vinyl ether and at least one cation is present in an amount of about 5 to about 55 wt. %, based on the total weight of the denture adhesive liner composition.

16. The method of claim 11, wherein said plasticizer is present in an amount of from 0 to about 30 wt. %, based on the total weight of the denture adhesive liner composition.

17. The method of claim 11, wherein said thermoplastic polymer component is present in an amount of from about 30 to about 90 wt. %, based on the total weight of the denture adhesive liner composition.

18. The method of claim 11, further including from about 0 to about 50 wt. %, based on the total weight of the denture adhesive composition, of additional materials selected from the group consisting of sodium carboxymethyl cellulose, polyvinyl pyrrolidone, polyvinyl alcohol, polyacrylic acid derivatives, sodium alginate, polyvinyl acetate, and mixtures thereof.

19. A single-layer denture adhesive liner prepared from the method of claim 11.

20. A multi-layer denture adhesive liner prepared from the method of claim 11 using a co-extruder.
